(19) United States
(12) Patent Application Publication
(10) Pub. No.: US 2008/0227854 A1
(43) Pub. Date: Sep. 18, 2008

(54) Compound 3-(1, 1-Dimethyl-Allyl)-6-Hydroxy-Chromen-2-One and its Pharmaceutically Acceptable Salts Thereof

(75) Inventors: Hasi Rani Das, Delhi (IN); Sunil Kumar Raghav, Delhi (IN); Bhawna Gupta, Delhi (IN)

Correspondence Address: DLA PIPER US LLP, 4365 EXECUTIVE DRIVE, SUITE 1100, SAN DIEGO, CA, 92121-2133, US

(73) Assignee: COUNCIL OF SCIENTIFIC & INDUSTRIAL RESEARCH, NEW DELHI (IN)

(21) Appl. No.: 11/935,985

(22) Filed: Nov. 6, 2007

(30) Foreign Application Priority Data
Mar. 8, 2007 (IN) 512/DEL/2007

(51) Int. Cl. A61K 31/352; C07D 311/04

(52) U.S. Cl. 514/456; 549/289

(57) ABSTRACT

Accordingly the present invention describes the anti-inflammatory compound 3-(1,1-dimethyl-allyl)-6-hydroxy-chromen-2-one isolated from the Ruta graveolens L. plant for the inhibition of inos gene thereby reducing nitric oxide level that increases significantly in inflammatory diseases. Thus the present invention relates to the discovery of lead compound for therapeutic intervention of the inflammatory diseases such as rheumatoid arthritis, systemic lupus erythrematosus, osteoarthritis and others, by the inhibition of inos gene and nitric oxide

### CROSS REFERENCE TO RELATED APPLICATION

This application is a utility application and claims the benefit under 35 USC § 119(a) of India Application No. 512/DEL/2007 filed Mar. 8, 2007. This disclosure of the prior application is considered part of and is incorporated by reference in the disclosure of this application.

### BACKGROUND OF THE INVENTION

1. Field of the Invention

The present invention relates to compound 3-(1,1-dimethyl-allyl)-6-hydroxy-chromen-2-one of formula I isolated from Ruta graveloens L.

The main utility of the invention is to provide lead molecule for development of new drugs for treating several inflammatory diseases in which nitric oxide plays an important role in inflammation.

2. Background Information

Inflammation is the body's reaction to invasion by an infectious agent, antigen challenge or even just physical, chemical or traumatic damage. Inflammatory conditions are characterized by generation of pathological concentration of nitric oxide due to increased expression of inos gene. All these conditions are associated with severe pain, discomfort and incapacitation in advanced case. The inducible NOS isoform is not expressed in normal conditions but can be induced in many types of cells such as chondrocytes, macrophages and fibroblast like synovial cells by inflammatory cytokines such as IL-1β and TNF-α or bacterial lipopolysaccharide (LPS) (Stadler et al., 1991, J Immunol. 147: 3915-20). Macrophages and synovial cells are the most abundant cell types in joints and they are the predominant source of nitric oxide in the inflamed synovium (McInnes I. B. et al., 1996, J. Exp. Med. 184, 1519-1524). Nitric oxide suppresses the cartilage proteoglycan synthesis by reducing the incorporation of sulphates into the glycosaminoglycans (GAGs) of cartilage proteoglycans. IL-1 induced nitric oxide also inhibits proliferation of chondrocytes (Taskiran et al., 1994, Biochem. Biophys. Res. Commun. 200, 142-148). Hence, nitric oxide is a potent contributor to the inflammation and related joint-damage observed in arthritic conditions. Recent studies suggest that inhibiting the inos gene may be an effective way to reduce inflammation.

Induced NO synthesis was reported in inflammatory responses initiated by microbial products or autoimmune reactions and also in the systemic inflammatory response, also referred to as sepsis. NO likely participates in the inflammatory reaction and subsequent joint destruction in some types of arthritis. For instance synovial fluid from patients with osteoarthritis exhibits elevated nitrate concentrations and nitrates are end products of the L-arginine-NO synthase pathway (Farrell, A. J. et al., 1992, Ann Rheum Dis. 51: 1219-22). There is also evidence for chronic expression of iNOS in the smooth muscle in atherosclerotic aortic aneurysms (Lee. J. K. et al., 2001, Arterioscler Thromb Vasc Biol. 21: 1393-401), a disease in which there is progressive dilatation and destruction of the aortic wall leading often to fatal rupture.

It is, therefore, important to diagnose and treat these inflammatory diseases at early stage to prevent irreversible damage of joints. For treating inflammation, several compounds belonging to coumarins and their derivatives have been described in the literature. But all of these compounds have their own limitations.

A large number of coumarins derivatives are known to inhibit the inflammatory condition by different mechanisms. Fraxetin, esculetin, 4-methylesculetin, daphnetin and 4-methyldaphnetin inhibited generation of leukotriene B4 (a 5-LO product) (Hoult et al., 1994a, Agents Actions 42: 44-49). Coumarin and umbelliferone were found to have a mechanism of action similar to NSAID in a carrageenan induced inflammation (Lino et al., 1997, Phytother. Res. 11: 211-215). Coumarin was also effective in the rat paw oedema induced by dextran. Osthol, isolated from Angelica archangelica, A. pubescens f. biserrata and Atractylodes lancea, turned out to be a selective inhibitor of 5-LO in vitro (Roos et al., 1997, Pharmacol. Lett. 7: 157-160; Liu et al., 1998, Planta Med. 64: 525-529; Resch et al., 1998, J. Nat. Prod. 61: 347-350). Since 5-LO is activated by calcium influx, this effect was suggested to be due to its calcium antagonistic properties (Harmala et al., 1992, Phytochem. Anal. 3: 42-48). Seselin from the aerial parts of Decatropis bicolor was active in the carrageenan-induced inflammation assay in rats (Garcia-Argaez et al., 2000, Planta Med. 66: 279-281). Carrageenan-induced rat paw oedema has been inhibited also by ethanol extract of the roots of Peucedanum ostruthium (Hiermann and Schlantl, 1998, Planta Med. 64: 400-403), 6-(3-carboxybut-2-enyl)-7-hydroxycoumarin being the most important anti-inflammatory compound in the plant. Carrageenan-induced inflammation was also suppressed by seselin isolated from Seseli indicum (Tandan et al., 1990, Fitoterapia LXI: 360-363) and by ethanol extract of the aerial parts of Ruta chalepensis (Al-said et al., 1990, J. Ethnopharmacol. 28: 305-312). Columbianadin, columbianetin acetate, bergapten and umbelliferone isolated from Angelica pubescens demonstrated both anti-inflammatory and analgesic activities at 10 mg/kg in mice (Chen et al., 1995, Planta Med. 61: 2-8.). Osthole and xanthotoxin revealed only anti-inflammatory activity, and isoimperatorin only analgesic effect. Interestingly, coumarins can also possess pro-inflammatory effects: lower doses of psoralen and imperatorin have shown an anti-inflammatory effect but at higher doses they have a pro-inflammatory effect (Garcia-Argaez et al., 2000, Planta Med. 66: 279-281).

Very few coumarins are known to inhibit the nitric oxide, an important mediator of inflammation. Inhibitor of nitric oxide production by N-Monomethyl L-arginine (L-NAME), which is a structural analogue of the iNOS substrate arginine, has been successfully used to block the nitric oxide mediated effects and appears to be chondro-protective in animal model (Guzik et al., 2003, Journal of Physiology and Pharmacology 54, 469-487).

It is evident from the above that need exists for development of new anti-inflammatory drugs which inhibits the inflammation through inhibition of nitric oxide production. Our very recent work (Raghav et al., 2006, J. Ethnopharmacol. 104: 234-239) on crude extract of Ruta graveolens showed inhibition of nitric oxide through inos gene inhibition on murine macrophage cells (J774) and crude extract is much more effective than pure rutin, a reported component in the plant. The novelty of the present invention is to provide a novel anti-inflammatory compound 3-(1,1-dimethyl-allyl)-6-hydroxy-chromen-2-one isolated from Ruta graveolens, which is effective at very low concentration and less cytotoxic as compared to rutin already reported from the plant.

### OBJECTS OF THE INVENTION

The main object of the present invention is to provide novel anti-inflammatory lead compound 3-(1,1-dimethyl-allyl)-6-hydroxy-chromen-2-one for developing new drugs for treating the inflammatory conditions.

Another object of the invention is to provide the process for isolation of the novel anti-inflammatory lead molecule 3-(1,1-dimethyl-allyl)-6-hydroxy-chromen-2-one from a plant Ruta graveolens.

Still another object of the present invention is to provide a method for testing the novel compound 3-(1,1-dimethyl-allyl)-6-hydroxy-chromen-2-one as an anti-inflammatory agent.

Yet another object of the invention is to provide usage of the novel compound 3-(1,1-dimethyl-allyl)-6-hydroxy-chromen-2-one as an anti-inflammatory compound.

The present invention relates to the anti-inflammatory compound 3-(1,1-dimethyl-allyl)-6-hydroxy-chromen-2-one of the formula I, that significantly inhibit (70%) inos gene, thereby inhibiting the nitric oxide produced, and therefore of use as a therapeutic agent for the treatment of several inflammatory diseases such as rheumatoid arthritis (RA), systemic lupus erythrymatosus (SLE) and others, thereby reducing joint inflammation and promoting normal cell growth.

The yet another aspect of the present invention is, use of the novel anti-inflammatory lead compound 3-(1,1-dimethyl-allyl)-6-hydroxy-chromen-2-one isolated from the diethyl ether fraction of the Ruta graveloens plant, as an inhibitor of nitric oxide and the inos gene leading to the suppression of LPS induced inflammatory condition. By inhibiting the inos gene, the isolated compound also promotes normal cell growth thereby reducing the cell damaging effects of the inflammatory diseases. The lead compound was also observed to inhibit the endotoxin induced inflammation or septic shock in Balb/c mice. It was also observed that the compound helped in keeping the normal behavioral condition of the mice.

### SUMMARY OF THE INVENTION

Accordingly the present invention describes the anti-inflammatory compound 3-(1,1-dimethyl-allyl)-6-hydroxy-chromen-2-one isolated from the Ruta graveolens L. plant for the inhibition of inos gene thereby reducing nitric oxide level that increases significantly in inflammatory diseases. Thus the present invention relates to the discovery of lead compound for therapeutic intervention of the inflammatory diseases such as rheumatoid arthritis, systemic lupus erythrematosus, osteoarthritis and others, by the inhibition of inos gene and nitric oxide

In an embodiment of the present invention, a compound 3-(1,1-dimethyl-allyl)-6-hydroxy-chromen-2-one, of general formula I. as given below

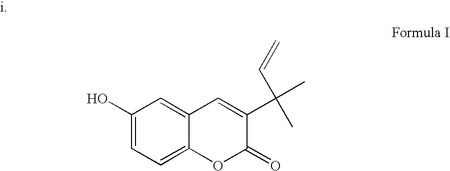

and its pharmaceutically acceptable salts thereof.

In yet another embodiment of the present invention, the purity of the said compound by HPLC is more than 94%.

In still another embodiment of the present invention, the said compound is isolated from the plant Ruta graveolens.

In an embodiment of the present invention, the compound is useful as anti-inflammatory agent.

In another embodiment of the present invention, the compound causes in-vitro 25%, 40% and 70% nitric oxide (NO) inhibition at a concentration of 5, 10 and 20 μg/ml respectively.

In yet another embodiment of the present invention, the compound inhibits in-vivo inflammation (NO inhibition) in the range of 60% to 80%, at the effective dose of 40 mg/kg to 160 mg/kg body weight.

In still another embodiment of the present invention, the cytotoxicity of the said compound, is within acceptable limits, as it is >85% at all the doses ranging from 20 to 100 μg/ml

In an embodiment of the present invention, the compound and its pharmaceutically acceptable salts are useful as a therapeutic agent.

In yet another embodiment of the present invention, the compound is useful in inhibiting inflammation.

In still another embodiment of the present invention, the compound as is useful in inhibiting inflammation by inhibition of nitric oxide (NO) production.

In an embodiment of the present invention, the compound is effective at very low concentration i.e., 5 μg/ml and less cytotoxic.

In another embodiment of the present invention, the compound and its pharmaceutically acceptable salts are useful for treatment of diseases selected from the group comprising of rheumatoid arthritis, systemic lupus erythrymatous and other inflammatory diseases in which nitric oxide plays an important role in pathogenesis or progression of the disease.

In yet another embodiment of the present invention, a pharmaceutical composition comprising compound 3-(1,1-dimethyl-allyl)-6-hydroxy-chromen-2-one, of general formula I., as given below

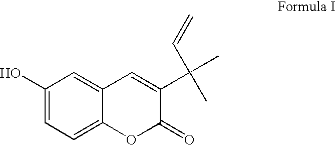

and its pharmaceutically acceptable salts thereof optionally along with a pharmaceutically acceptable carrier or an additive.

In still another embodiment of the present invention, the pharmaceutical composition is in the form of powder, injectible, syrup, capsule, or a tablet

In an embodiment of the present invention, a process for isolation of compound, from the plant Ruta graveolens, wherein the said process comprising:
a. extracting the dried Ruta graveolens plant with an organic solvent at room temperature;
b. removing the solvent from the extract obtained from step a);
c. re-extracting the residue obtained from step (b) with diethyl ether (the solvents having comparable polarity to diethyl ether can be used), and removing the diethyl ether to obtain residue;
d. screening of the residue obtained from step (c) for inhibition of nitric oxide production;
e. purifying the active compounds from the residue obtained in step (d), using reverse phase-HPLC, and assaying all the peaks obtained for the inhibition of NO production;
f. characterizing and identifying the isolated purified compound obtained in step (e) using analytical techniques like ESI-MS/MS, MALDI-TOF, FT-IR and NMR.

In another embodiment of the present invention, the process for isolation of compound wherein the solvent used for extraction is selected from the group consisting of alcoholic solvent preferably methanol, ketonic solvents preferably acetone and diethyl ether.

### BRIEF DESCRIPTION OF THE DRAWINGS

FIG. 1 Griess nitrite assay showing the inhibition of nitric oxide.

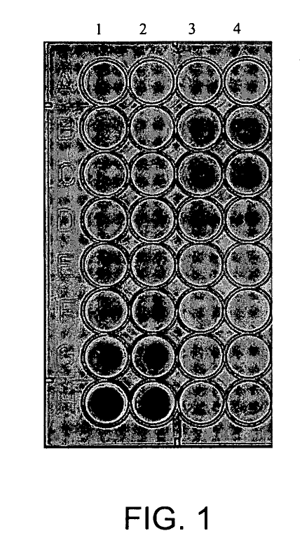

Lane 1-2 (A-H): Standard sodium nitrite (in duplicate)

Lane 3-4 (A): Unstimulated control (in duplicate)

Lane 3-4 (B): LPS Challenged (in duplicate)

Lane 3-4 (C-E): Lead compound treated (5, 10, 20 ug/ml) and LPS challenged (in duplicate)

FIG. 2. Nitric oxide inhibition measured as nitrite concentration using Griess nitrite assay in culture supernatant of macrophage cells (J774). Cells preincubated for 2 hours with ethyl ether fraction and the purified compound 3-(1,1-dimethyl-allyl)-6-hydroxy-chromen-2-one, isolated from diethyl ether fraction of Ruta graveolens plant and then challenged with LPS for 16 hours. Cells were challenged with only LPS (16 hours) as positive control. * p<0.05, ** p<0.01

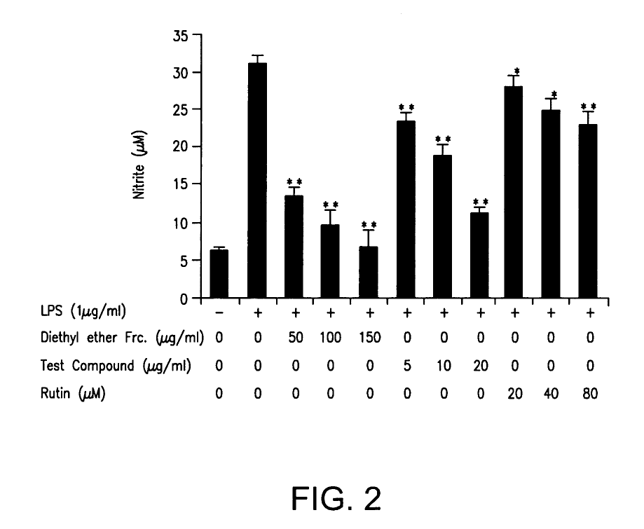

FIG. 3 (A) inos gene expression in murine macrophages (J774) preincubated for 2 hours with ethyl ether fraction and lead compound, 3-(1,1-dimethyl-allyl)-6-hydroxy-chromen-2-one isolated from the diethyl ether fraction of Ruta graveolens plant followed by LPS challenge (4 hours). The labeling present below the bar graph for densitometric analysis also corresponds to the bands present in the agarose gel, (B) Densitometric analysis of the bands observed in the agarose gel.

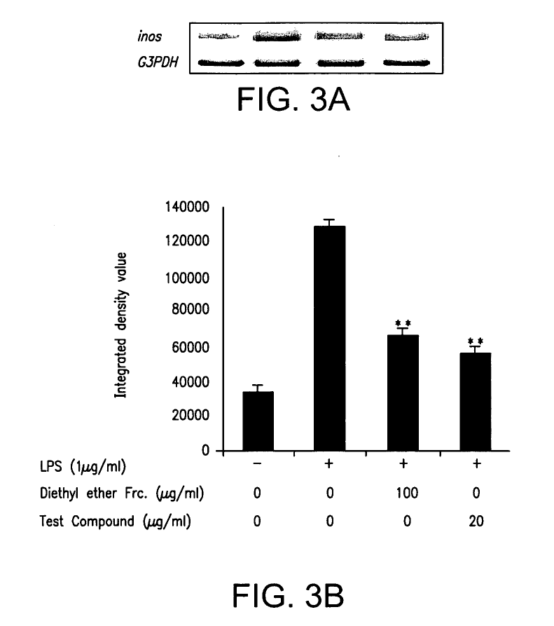

FIG. 4 Nitric oxide inhibition measured as total nitrite in plasma samples of LPS (150 μg) challenged Balb/c mice (8 hours). The mice (treated group) were preinjected (i.p.) for 2 hours with the active compound, 3-(1,1-dimethyl-allyl)-6-hydroxy-chromen-2-one at a dose of 4 mg/mice (160 mg/kg body weight) before LPS challenge. The blood samples were collected after 8 hours of LPS challenge.

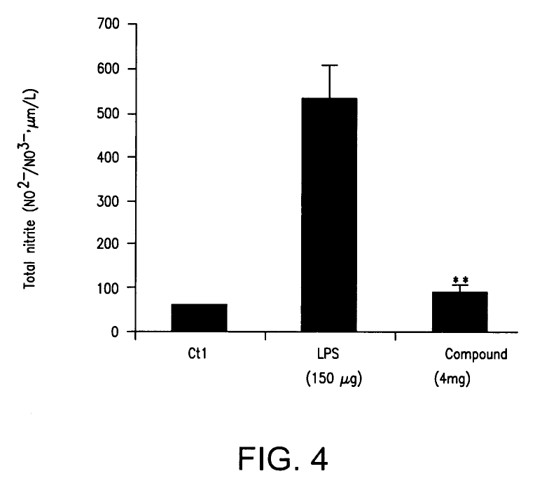

FIG. 5 Reverse phase HPLC analysis of ethyl ether fraction containing the active compound 3-(1,1-dimethyl-allyl)-6-hydroxy-chromen-2-one.

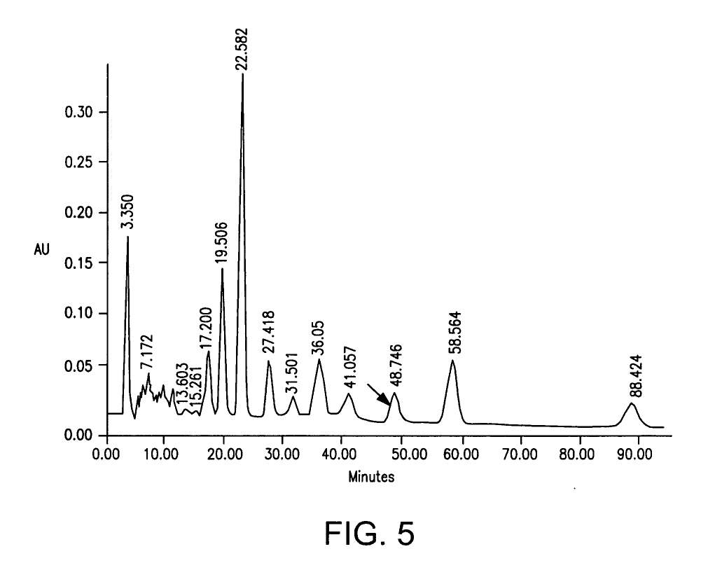

FIG. 6 Reverse phase HPLC analysis of purified active compound 3-(1,1-dimethyl-allyl)-6-hydroxy-chromen-2-one for purity checking.

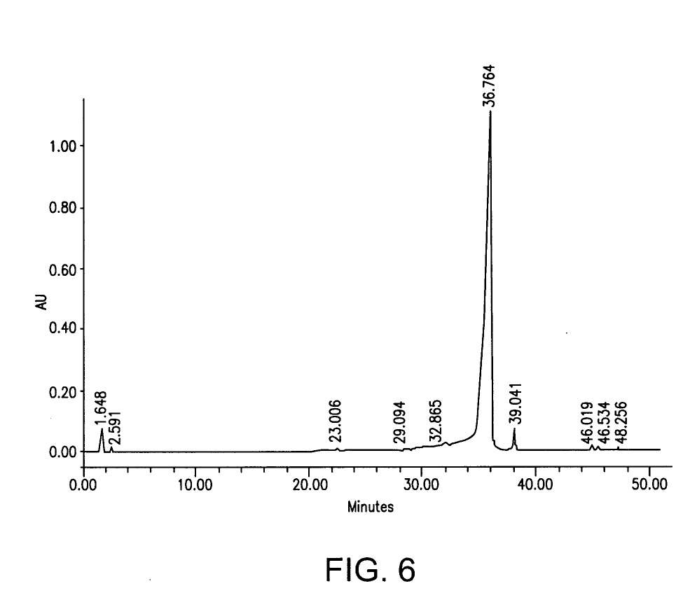

FIG. 7 Electron spray ionization-mass spectroscopy/mass spectroscopy (ESI-MS/MS) spectra of 3-(1,1-dimethyl-allyl)-6-hydroxy-chromen-2-one.

FIG. 8 Matrix assisted laser desorption and ionization-time of flight/time of flight (MALDI-TOF/TOF) spectra of 3-(1,1-dimethyl-allyl)-6-hydroxy-chromen-2-one.

FIG. 9 Infrared spectrum of lead compound 3-(1,1-dimethyl-allyl)-6-hydroxy-chromen-2-one isolated from the diethyl ether fraction of Ruta graveolens plant.

FIG. 10 1H Nuclear Magnetic Resonance Spectra of 3-(1,1-dimethyl-allyl)-6-hydroxy-chromen-2-one.

FIG. 11 ¹³C Nuclear Magnetic Resonance Spectra of 3-(1,1-dimethyl-allyl)-6-hydroxy-chromen-2-one.

FIG. 12 2-D COSY Spectra of 3-(1,1-dimethyl-allyl)-6-hydroxy-chromen-2-one.

### DETAILED DESCRIPTION OF THE INVENTION

### Preparation of 50% Methanolic Extract

Plant material Ruta graveolens (Rutaceae) was collected during the month of December/January from the Homeopathic Pharmacopeia Laboratory (HPL) herbal garden, Ghaziabad and authenticated by Dr. Prakash Joshi (senior scientific officer, HPL, India). This laboratory is responsible for authentication of all the plants and plant products used in homeopathic medicine in India. The dried material was powdered and extracted with 50% methanol. All the extract obtained after three cycles of extraction with methanol were pooled and methanol was evaporated under reduced pressure at 45° C. in Rota-vapor (Buchi) attached to a waterbath. The water from the extract was then lyophilized using freeze drier (Vertis). The residue obtained was stored at 4° C. till use. The 50% methanolic extract was examined for its anti-inflammatory effects in murine J-774 macrophages (Raghav et al., 2006, J. Ethnopharmacol. 104: 234-239). The extract was then subjected to further fractionation for isolation of the active anti-inflammatory compounds.

(2) Fractionation of 50% Methanolic Extract of Ruta graveolens L.

The constituents of the crude 50% methanolic extract were fractionated using solvent extraction method. The dried compound obtained by 50% methanol extraction was suspended in distilled water and different compounds were separated by successive exhaustive extraction with diethyl ether, chloroform and ethyl acetate respectively with increasing polarity. After complete removal of solvents all the fractions were tested for their effect on nitric oxide production using murine macrophages stimulated with lipopolysaccharide (LPS) from E. Coli (strain 055:B5). The diethyl ether fraction, which showed inhibitory effect, was then sub-fractionated using RP-HPLC.

### (3) Reverse Phase-High Performance Liquid Chromatography (RP-HPLC)

First, the active diethyl ether fraction was passed through 0.22 μm filter and then loaded on to pre-equilibrated analytical Sunfire™ C₁₈ column (PDA detector, waters system, pump 600e) at room temperature. The isocratic mobile phase containing 30% acetonitrile and 70% MilliQ water with 0.05% TFA was used. The flow rate was maintained at 1 ml/min. Thirteen well separated peaks were obtained. Then, preparative Sunfire™ C₁₈ column was used for collecting the individual compounds (peak material) in sufficient quantity. The same isocratic mobile phase was used for the collection of the individual compounds/peaks. The acetonitrile was then evaporated by keeping the fractions overnight at 45° C. over a water bath and the water was evaporated using freeze drier. The residue obtained from each fraction was properly labeled and stored at 4° C. till use. All these thirteen peak materials were screened for inhibition of NO production in macrophage cells (J774) challenged with LPS. The compound showing significant inhibitory effect was subjected to rerun RP-HPLC column using gradient mobile phase to confirm its purity.

### Characterization Studies

### (1) Structure Elucidation

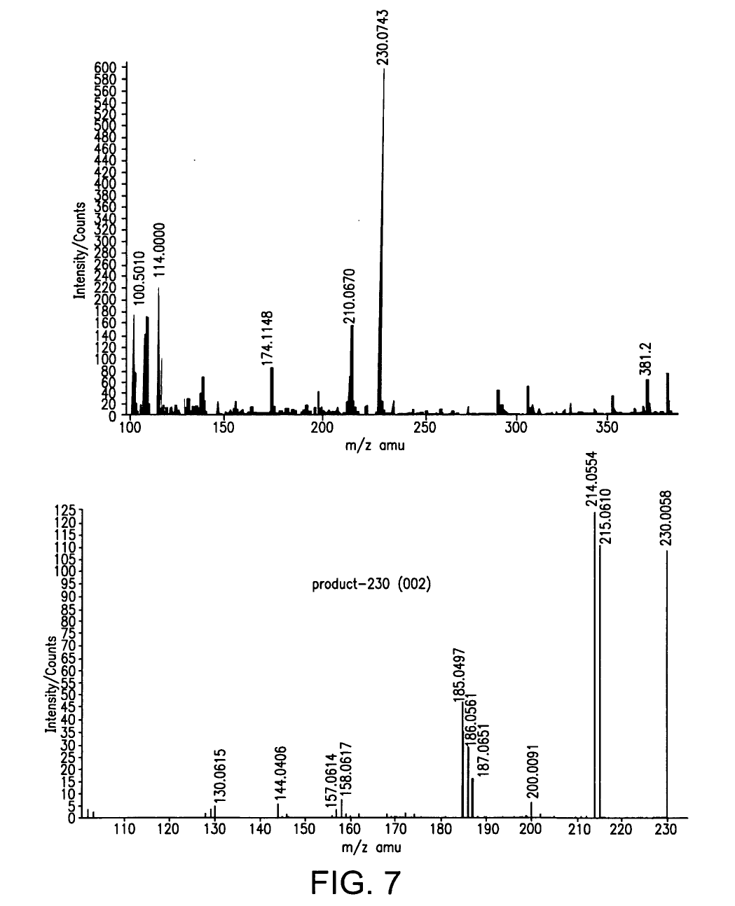

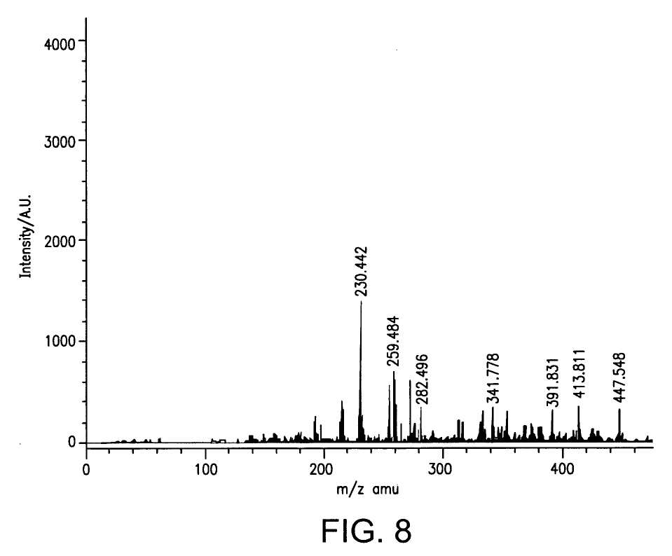

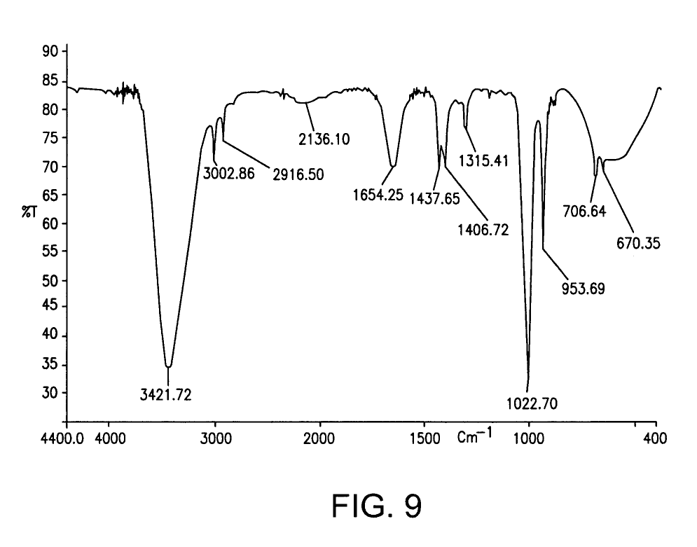

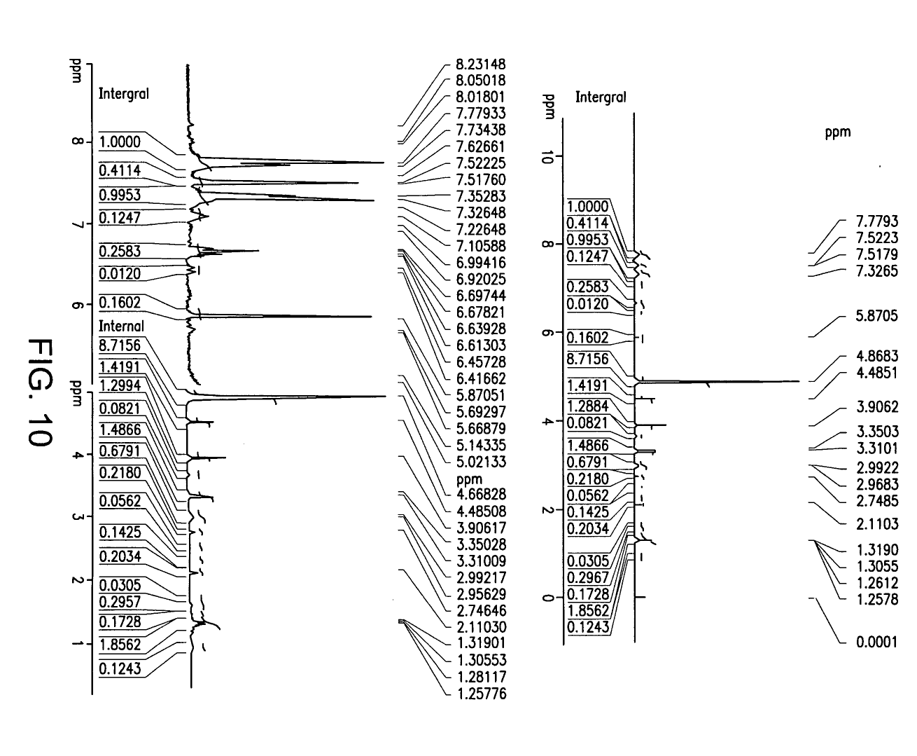

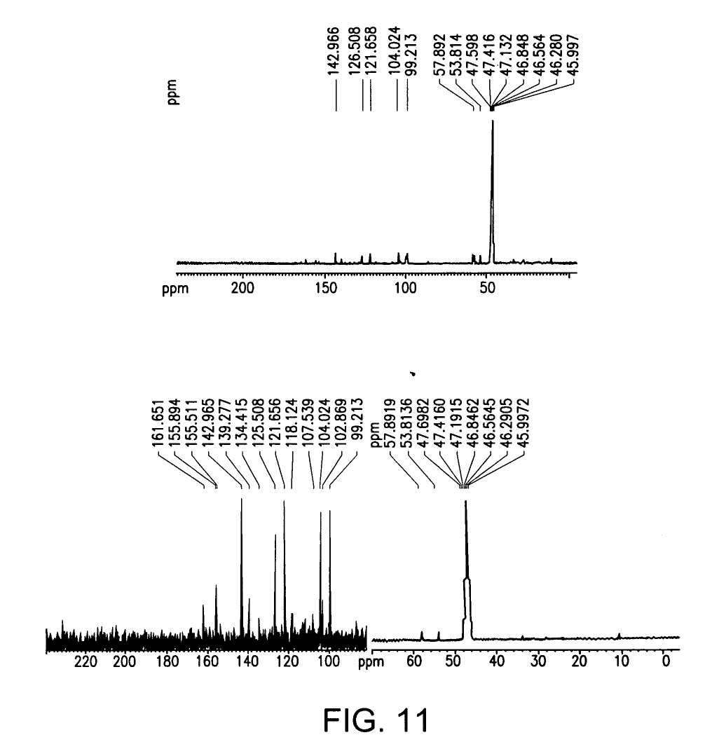

Characterization of the compounds was carried out using various techniques such as infra-red spectrum (FIG. 9), mass spectroscopy (FIGS. 7, and 8), and nuclear magnetic resonance spectroscopy (FIGS. 10 and 11). Details of the spectral data obtained with respect to novel compound are as described earlier.

### Anti-Inflammatory Activity Assays

The anti-inflammatory activity of various fractions such as crude methanolic extract, sub-fractions and purified compounds was studied by nitric oxide assay using Griess nitrite assay, reverse transcription-polymerase chain reaction (RT-PCR) and in LPS induced endotoxemia in Balb/c mice.

### (1) Cell Culture

Murine macrophage cell line (J-774) was used to check the anti-inflammatory effects of the extract, sub-fractions and the isolated active compounds. The macrophages are cultured in Dulbeccos Modified Eagles Medium (DMEM) supplemented with antibiotic and antimycotic solution and 10% fetal calf serum at 37° C. in a CO₂ incubator (5%). The cell viability was measured using the Trypan blue exclusion method. For inducing the inflammatory conditions the cell were stimulated with LPS (1 μg/ml) and the effect of the compounds on the inflammatory condition was assessed by measuring the inflammatory mediator, nitric oxide and the respective inos gene expression by the murine macrophages.

### (2) Nitric Oxide Assay Using Griess Nitrite Assay

The murine macrophage cells release nitric oxide along with other pro-inflammatory molecules, when stimulated with LPS. The cells were preincubated with the crude extract (300 and 500 μg/ml), ethyl ether fraction (50, 100, 150 μg/ml), the isolated active compound (5, 10, 20 μg/ml) and rutin (20, 40, 80 μM) for 2 hours and then challenged with LPS (1 μg/ml) for 16 hours. After 16 hours the cell supernatant was used to measure the nitric oxide production as nitrite using Griess nitrite assay (Lee et al., 2003, Life Science 73, 1401-1412). Equal amount of cell supernatant was incubated with Griess reagent for 10 minutes at room temperature and the magenta color developed was measured using spectrophotometer.

### (3) RT-PCR Analysis for Inos Gene Expression

The murine macrophage cells were preincubated with the test extract, ethyl ether fraction and the compound for 2 hours (as mentioned above), washed and then challenged with LPS for 4 hours. After the incubation period the cells were washed with phosphate buffered saline (PBS), scraped and then pelleted by centrifugation. The total RNA from the pelleted cells was isolated using EZ-RNA isolation kit (Biological industries) using vendor recommended protocol. Then, cDNA was prepared from the total RNA using single strand cDNA synthesis kit (Clontech, USA) and the expression of inos gene was estimated using gene specific primers for inos gene.

(4) Cytotoxicity assay using MTT [3-(4,5-dimethylthiazol-2-yl)-2,5-diphenyltetrazolium bromide]

The murine macrophage cells were plated in cell culture plates and then treated with or without the crude extract/ethyl ether fraction/purified compound. The cells were then washed and challenged with LPS (1 μg/ml) for 16 hours. The MTT (5 mg/ml in PBS) was then added and the cells were further incubated for 4 hours at 37° C. in CO₂ incubator. MTT solubilization buffer was then added and absorbance at 590 nm was determined. It was observed that the highest concentrations of each extracts, such as 50% methanolic extract (500 μg/ml), ethyl ether fraction (200 μg/ml) and the purified lead compound (100 μg/ml) were showing >85% viability.

The Efficacy and Toxicity of
3-(1,1-dimethyl-allyl)-6-hydroxy-chromen-2-one in
Comparison to Rutin

| | | | Nitric oxide | Cell viability |
|---|---|---|---|---|
| | Conc. (μg) | Conc. (μM) | inhibition (%) | (%) |
| Rg-001 | 5 | 21.74 | 25.4 | >85 |
| | 10 | 43.5 | 40 | >85 |
| | 20 | 87 | 63.5 | >85 |
| Rutin | 12.2 | 20 | 11 | >85 |
| | 24.4 | 40 | 20.6 | >85 |
| | 48.8 | 80 | 27 | ≦75 |

The viability of macrophage cells was >85% at highest concentration (50 μg i.e. 217.5 μM) of 3-(1,1-dimethyl-allyl)-6-hydroxy-chromen-2-one tested using MTT cell viability assay whereas for rutin the cell viability decreased significantly at higher concentration (80 μM) and above. The isolated compound 3-(1,1-dimethyl-allyl)-6-hydroxy-chromen-2-one was found to be more efficacious (63.5% inhibition at 87 μM conc.) and less toxic (>85% viability) then rutin which showed 27% inhibition at 80 μM conc. and ≦75 cell viability (more toxic) for nitric oxide inhibition.

(5) Anti-Inflammatory Activity of the Pure Compound 3-(1,1-dimethyl-allyl)-6-hydroxy-chromen-2-one, In Vivo in Balb/c Mice

The HPLC purified lead compound was suspended in phosphate buffered saline (PBS) at a concentration of 20 mg/ml for in vivo experiment. Balb/c mice, 6 to 8 week of age, weighing 23-25 g, were housed in micro-barrier cages on sterile bedding and fed ad libitum water and food. The animals were divided into three groups containing 6 mice in each group. Four milligrams (160 mg/kg body weight) of the isolated lead compound was administered intra-peritoneally (i.p.) 2 hours prior to LPS challenge. After 2 hours 150 μg LPS was injected i.p., and the mice were kept for 8 hours. In negative control mice, PBS was injected whereas, only LPS injected mice were used as positive control. After 8 hours of LPS injection, the blood was collected in ACD buffer from each mouse by retro-orbital puncture. The blood samples were immediately centrifuged and the plasma samples were collected and stored at −20° C. till nitrite assay. The plasma samples were then diluted 1:1 using 1× reaction buffer (total nitrite estimation kit, R & D systems, USA) and protein from the plasma was removed using 10 kDa centricon (Centricon, USA). Then protein free plasma samples were used for total nitrite estimation (NO²⁻+NO³⁻) using the same kit. The compound was observed to significantly inhibit the total nitrite in the plasma samples (>75%). The treated mice were observed to be in normal behavioral condition as compared to the LPS challenged mice.

The invention is illustrated by the following examples, which are provided to illustrate the invention and should not be construed as limitation in the inventive concept herein.

### EXAMPLE I

### Procurement of Ruta graveolens L. Plant

The plant Ruta graveolens L. was procured from and authenticated by Dr. Prakash Joshi (senior scientific officer) Homeopathic Pharmacopoeia Laboratory (HPL), Ghaziabad, India (This national laboratory is responsible for the validation of all the plants and plant products used in Homeopathy). The whole plant was collected during the month of December-January in the year 2002-2003 from HPL herbal garden.

### EXAMPLE II

### Preparation of 50% Methanol Extract of Ruta graveolens L. Plant

In the present invention, the plant extract of Ruta graveolens L. was prepared using solvent extraction method. First the whole plant was vacuumed dried and grinded. The grinded plant is weighed, submerged in 50% methanol in distilled water and soaked for two days. The solution was filtered through Whatman filter paper No. 1 and then filtered through 0.22 μm filter. The filtered extract was vacuumed dried, weighed and stored in an airtight container.

### EXAMPLE III

### Solvent Fractionation of 50% Methanol Extract of Ruta graveolens L. Plant

The 50% methanol extract of the plant was dried, weighed and suspended in adequate amount of de-ionized water in a conical flask. The flask was put on shaking till a uniform suspension was obtained. The suspension was transferred to a separating funnel for “solvent fractionation”. The extract was then exhaustively fractionated using different organic solvents (Ethyl ether<Chloroform<Ethyl acetate) according to their increasing polarity. The organic solvents were then evaporated using rotary evaporator and the samples were lyophilized using freeze drier. The dried powder recovered from each fraction was weighed and dissolved in dimethylsulfoxide (DMSO) at a concentration of 50 mg/ml.

### EXAMPLE IV

### Analysis of Rutin and Quercetin in the Ethyl Ether Fraction by Reverse Phase HPLC

Rutin and Quercetin (Sigma) was added to the ethyl ether fraction as internal standards to confirm that these were either absent or negligibly present in the said fraction.

### EXAMPLE V

### Screening of Fractions for Nitric Oxide Inhibition In Vitro

| Fraction used | Concentration (μg/ml) | Inhibition (%) |
|---|---|---|
| Diethyl ether fraction | 100 | 69.8 |
| Chloroform fraction | 100 | 10.5 |
| Ethyl acetate fraction | 100 | 13.2 |
| Water Fraction | 100 | 8.6 |

Each of the four fractions obtained in Example III were then used in different concentrations (50 μg/ml, 100 g/ml and 150 μg/ml) for their effect on nitric oxide inhibition in vitro. Murine macrophages (J774) were maintained in DMEM medium, 37° C. in CO2 incubator (5% CO2). The cells were harvested and challenged with LPS (Sigma) at a concentration of 1 μg/ml with and without the four test fractions obtained in Example III for 16 hours at 37° C. in the humidified incubator with 5% CO2. After 16 hours the cell free supernatant was collected and the nitric oxide production was measured as described by Lee et al. Briefly 100 μl of supernatant samples were incubated for 10 min at room temperature with an equal volume of Griess reagent (0.1% naphthalene diamine dihydrochloride, 1% sulfanilamide in 5% H₂SO₄) in microtitre plate. The absorbance at 550 nm was measured. Sodium nitrite was used as the standard.

### EXAMPLE VI

### Screening of Fractions for Inos Gene Inhibition In Vitro

The murine macrophage cells (J774) were challenged with LPS at a concentration of 1 μg/ml with and without the four test fractions obtained in Example III for 4 hours at 37° C. in the humidified incubator with 5% CO₂. The cells were harvested and were used to isolate the total RNA using (RNA isolation kit, Qiagen). The RNA was quantified spectrophotometrically and 2 μg of total RNA from each sample was used for cDNA synthesis (cDNA synthesis kit, Clontech). Gene specific PCR for the inducible nitric oxide synthase (inos) and glyceraldehyde-3-phosphatedehyrogenase (G3PDH) as house keeping gene was performed. Primer sequences were designed from cDNA sequences of the specific gene (www:ncbi.nlm.nih.gov/locuslink) using DNASTAR software. The primer sequences used to amplify the inos gene were: forward primer-5′TCACTGGGACAGCACAGAAT3′ and reverse primer-5′TGTGTCTGCAGATGTGCTGA3′. PCR mixture consist of 25 mM 10×Taq buffer containing 15 mM Mg²⁺, 5 mM dNTPs, 10 pM each of the forward and the reverse primers, 2 units Taq DNA polymerase enzyme and 2 μl of 1:5 diluted cDNA for a 25 μl reaction. PCR conditions to amplify the inos gene included an initial denaturation at 94° C. for 4 min and 34 cycles at 94° C. for 30 s, 60° C. for 30 s, 72° C. for 1 min and then a final extension for 3 min at 72° C. For the amplification of the G3PDH gene the PCR conditions followed, an initial denaturation at 94° C. for 4 min, 29 cycles at 94° C. for 45 s, 60° C. for 45 s, 72° C. for 2 min and then a final extension for 7 min at 72° C. The amplified PCR products of inos and G3PDH gene (510 bp and 983 bp respectively) were ran on 1.2% agarose gel and the densitometric analysis of the gene specific PCR products with respect to G3PDH gene was carried out using Digidoc 1201 software.

### EXAMPLE VII

### Statistical Analysis

The significance of differences from the respective controls was tested using student's T-test for each paired experiment. P≦0.05 was considered as significant. ** indicates p<0.01 and * indicates p<0.05.

(The ** and * indicates that the effect of the compound is very significant and significance increases as the p value decreases from 0.05.)

### EXAMPLE VIII

### Isolation of Lead Compound from Ethyl Ether Fraction of Ruta graveolens L Plant

After screening of different solvent fractions, the ethyl ether fraction of the plant was found to give significant nitric oxide inhibition. The active components from the ethyl ether fraction that inhibit nitric oxide were separated by reversed phase HPLC (Pump 600e, PDA detector, waters, USA). The photodiode array detector was used for detection of eluted compounds. The method was developed for the proper separation of individual peaks from ethyl ether fraction using analytical Sunfire™ C₁₈ (5 μm; 4.6×150 mm) column. The isocratic mobile phase (70% milliQ water with 0.05% TFA and 30% acetonitrile) was observed to give 13 peaks with proper separation. Each run was standarized for ninety minutes. Ten micrograms (10 μl) of sample was injected. Sunfire™ C₁₈ semi-preparative column (10 μm; 10×250 mm) was used for collection of individual peaks in sufficient amount. The same isocratic mobile phase (70% milliQ water containing 0.05% TFA and 30% acetonitrile) was used and the run time was ninety minutes, as all the 13 peaks were completely resolving in the specified time. Two hundred microlitres of injection volume containing 20 mg of ethyl ether fraction was injected for collection of individual peak/material in separate conical flasks. The acetonitrile from the eluted fractions was evaporated by leaving the samples overnight at 50° C. in water bath and the samples were finally lyophilized using freeze drier at −80° C. The dried samples were weighed and stored at 4° C. in micro centrifuge tubes. From each purified sample 2.5 mg/ml of stock was prepared in DMSO for in vitro screening for nitric oxide inhibition.

### EXAMPLE IX

### Screening of Fractions for Nitric Oxide and Inos Gene Inhibition In Vitro

Thirteen fractions collected from the diethyl ether fraction by reverse phase HPLC were screened for their effect on nitric oxide production. The inhibitory effect of rutin (20, 40 and 80 μM) on nitric oxide was measured to compare the inhibitory effect of isolated fractions (as rutin is present in rue and reported to inhibit the nitric oxide) present in the plant. The method used for the screening was similar to as described in Example III and Example V. It was observed that the fraction XI was showing significant inhibition of nitric oxide production at concentrations of 5, 10 and 20 μg/ml. Subsequently the above active fraction was examined for its effect on inos gene expression and found to inhibit the gene expression significantly (Table is not required as out of the thirteen fractions obtained, only the fraction XI was observed to inhibit the nitric oxide inhibition and then studied further for its effect and inhibition on inos gene inhibition).

### EXAMPLE X

### In Vivo Experiment for the Anti-Inflammatory Effect of the Active Compound 3-(1,1-DIMETHYL-ALLYL)-6-HYDROXY-CHROMEN-2-ONE on LPS Induced Endotoxemia

The Balb/c mice were used to validate the in vivo effect of the active compound on endotoxin induced inflammation by LPS. Balb/c mice, 6 to 8 week of age, weighing 23-25 g, were housed in micro-barrier cages on sterile bedding and fed ad libitum water and food. The animals were divided into three groups containing 6 mice in each group. Four milligrams (160 mg/kg body weight) of the active compound was preinjected intra-peritonially (i.p.) into the mice for two hours and then LPS (150 μg) was injected i.p. and left for eight hours. After eight hours the normal behavior of the treated and untreated mice was observed blindly by a volunteer. The blood was then drawn from each mice using retro-orbital puncture and collected in ACD buffer. The plasma from the blood samples were collected by centrifuging at 200 rpm for 10 minutes and stored at −80° C. till nitric oxide was analyzed using Griess nitrite assay. The total nitrite in the plasma samples were detected using nitric oxide (NO²⁻/NO³⁻) assay kit (R & D systems, USA). The plasma were first given a 10 kDa cut using 10 kDa centricon after diluting 1:1 using 1× reaction buffer to remove most of the proteins. The total nitrite was then estimated in the protein free plasma samples using the vendor recommended protocol.

### EXAMPLE XI

### Structural Elucidation of the Compound

Correlating all the spectral and chemical analysis information of the compound, the applicants have carried out the characterization of the compound.

(i) Electron spray ionization-mass spectroscopy/mass spectroscopy (ESI-MS/MS): This technique was used to determine the molecular weight of the lead compounds. The instrument used was ESI-LC-MS/MS from Bruker Daltonics. The compound sample was prepared in MS grade methanol at a concentration of 100 μg/ml. The molecular weight of the lead compound was observed to be 230 amu. (FIG. 7).

(ii) Matrix associated laser desorption and ionization (MALDI-TOF/TOF): MALDI-TOF/TOF (Bruker Daltonics, USA) was performed to confirm the molecular mass of the lead compound. The sample was prepared by mixing sample in the matrix di-hydroxy benzoic acid in equal proportions and the sample were then spotted on the chip and dried before analysis. It was observed that the mass of the compound correspond to mass as observed by ESI-MS/MS (FIG. 8).

(iii) Fourier Transform-Infrared Spectroscopy (FT-IR): This technique was used to get an idea about the functional groups present in the lead compound. The instrument used was spectrum BX series. Potassium bromide (KBr) disc method was used for sample preparation.

By examining a large number of infrared spectrums (reported in literature) of known organic compounds containing functional groups, we established the functional groups present in the compound. The correlation data (correlation charts from William Kemp) was also used to deduce the functional groups present in the lead compound (FIG. 9).

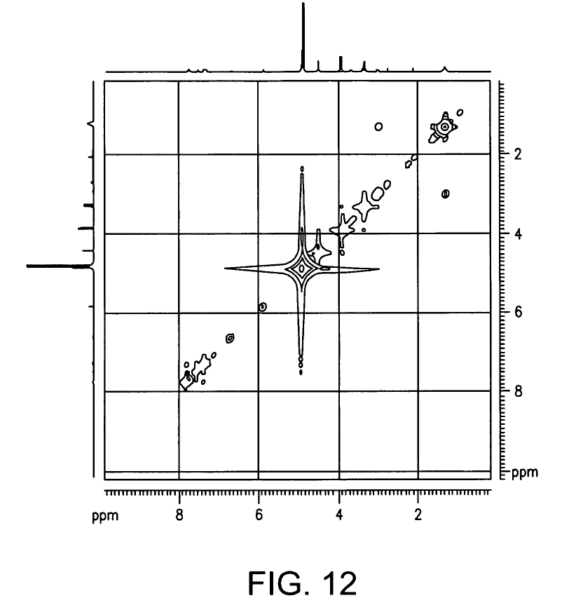

(iv) Nuclear Magnetic Resonance Spectroscopy (NMR): The NMR technique is used for structure analysis of the compound. The various analysis performed was ¹H-NMR, ¹³C-NMR and 2D-COSY. The instrument used was Avance 300 MHz (Bruker Daltonics, USA) (FIGS. 10, 11 and 12). The sample (15 mg) was dissolved in deuterated methanol (500 μl) and taken in sample tubes used for NMR analysis. The structures of the lead compounds were interpreted using NMR spectrum and the deduced structure of the lead compound is presented in the figure (FIG. 13) as formula I. The IUPAC name of the novel anti-inflammatory compound is 3-(1,1-dimethyl-allyl)-6-hydroxy-chromen-2-one.

### ADVANTAGE OF THE INVENTION

The present invention relates to novel anti-inflammatory compound 3-(1,1-dimethyl-allyl)-6-hydroxy-chromen-2-one isolated from Ruta graveloens L plant.

The isolated novel anti-inflammatory compound 3-(1,1-dimethyl-allyl)-6-hydroxy-chromen-2-one inhibits nitric oxide mediated inflammation both in vitro and in vivo.

The main utility of the present invention is to provide lead molecule for development of new drugs for treating inflammatory diseases in which nitric oxide plays an important role in inflammation.

Therefore the isolated novel anti-inflammatory compound 3-(1,1-dimethyl-allyl)-6-hydroxy-chromen-2-one is of use as a therapeutic agent for the treatment of several inflammatory diseases such as rheumatoid arthritis (RA), systemic lupus erythrymatosus (SLE) and others, thereby reducing joint inflammation and promoting normal cell growth

### REFERENCES

Stadler, J., Stefanovic-Racic, M., Billiar, T. R., Curran, R. D., McIntyre, L. A., Georgescu, H. I., Simmons, R. L., Evans, C. H., (1991). Articular chondrocytes synthesize nitric oxide in response to cytokines and lipopolysaccharide. J Immunol. 147: 3915-20.
McInnes, I. B., Leung B. P., Field, M., Wei, X. Q., Huang, F. P., Sturrock, R. D., Kinninmonth, A., Weidner, J., Mumford, R., Liew, F. Y., (1996). Production of nitric oxide in the synovial membrane of rheumatoid and osteoarthritis patients. J Exp Med. 84: 1519-24.
Taskiran, D., Stefanovic-Racic, M., Georgescu, H., Evans, C., 1994. Nitric oxide mediates suppression of cartilage proteoglycan synthesis by interleukin-1. Biochemical and Biophysical Research Communication 200, 142-148.
Farrell, A. J., Blake, D. R., Palmer, R. M., Moncada, S., (1992). Increased concentrations of nitrite in synovial fluid and serum samples suggest increased nitric oxide synthesis in rheumatic diseases. Ann Rheum Dis. 51: 1219-22.
Lee, J. K., Borhani, M., Ennis, T. L., Upchurch, G. R. Jr., Thompson, R. W., (2001). Experimental abdominal aortic aneurysms in mice lacking expression of inducible nitric oxide synthase. Arterioscler Thromb Vasc Biol. 21: 1393-401.
Hoult, J. R. S., Forder, R. A., Heras, B., Lobo, I. B. and Paya, M., (1994a). Inhibitory activity of a series of coumarins on leukocyte eicosanoid generation. Agents Actions 42: 44-49.
Lino, C. S., Taveira, M. L., Viana, G. S. B. and Matos, F. J. A., (1997). Analgesic and antiinflammatory activities of Justicia pectoralis Jacq and its main constituents:coumarin and umbelliferone. Phytother. Res. 11: 211-215.
Roos, G., Waiblinger, J., Zschocke, S., Liu, J. H., Klaiber, I., Kraus, W. and Bauer, R. (1997): Isolation, identification and screening for COX-1- and 5-LO-inhibition of coumarins from Angelica archangelica. Pharm. Pharmacol. Lett. 7: 157-160.
Liu, J. H., Zschocke, S., Reininger, E. And Bauer, R. (1998): Inhibitory effects of Angelica pubescens f. biserrata on 5-lipoxygenase and cyclooxygenase. Planta Med. 64: 525-529.
Resch, M., Steigel, A., Chen, Z. L. and Bauer, R. (1998): 5-Lipoxygenase and cyclooxygenase-1 inhibitory active compounds from Atractylodes lancea. J. Nat. Prod. 61: 347-350.
Harmala, P., Vuorela, H., Nyiredy, S. Z., Tornquist, K., Kaltia, S., Sticher, O. and Hiltunen, R. (1992): Strategy for the isolation and identification of coumarins with calcium antagonistic properties from the roots of Angelica archangelica. Phytochem. Anal. 3: 42-48.
Garcia-Argaez, A. N., Apan, T. O. R., Delgado, H. P., Velazquez, G. and Martinez-vazquez, M., (2000). Anti-inflammatory activity of coumarins from Decatropis bicolor on TPA ear mice model. Planta Med. 66: 279-281.
Hiermann, A. and Schantl, D., (1998). Antiphlogistic and antipyretic activity of Peucedanum ostruthium. Planta Med. 64: 400-403.
Tandan, S. K., Chandra, S., Tripathi, H. C. and Jawahar Lal, (1990). Pharmacological actions of seselin, a coumarin from Seseli indicum seeds. Fitoterapia LXI: 360-363.
Al-said, M. S., Tariq, M., Al-yahya, M. A., Rafatullah, S., Ginnawi, O. T. and Ageel, A. M., (1990). Studies on Ruta chalepensis, an ancient medicinal herb still used in traditional medicine. J. Ethnopharmacol. 28: 305-312.
Chen, Y. F., Tsai, H. Y. and Wu, T. S., (1995). Anti-inflammatory and analgesic activities from the roots of Angelica pubescens. Planta Med. 61: 2-8.
Guzik, T. J., Korbut, R., Adamek-Guzik, T., (2003). Nitric oxide and superoxide in inflammation and immune regulation. Journal of Physiology and Pharmacology 54, 469-487.
Lee, D. U., Kang, Y. J., Park, M. K., Lee, Y. S., Seo, H. G., Kim, T. S., Kim, C. H., Chang, K. C., (2003). Effects of 13-alkyl-substituted berberine alkaloids on the expression of COX-II, TNF-α, iNOS and IL-12 production in LPS-stimulated macrophages. Life Science 73, 1401-1412.
Anti-inflammatory effect of Ruta graveolens L. in murine macrophage cells. Raghav S K, Gupta B, Agrawal C, Goswami K, Das H R. J Ethnopharmacol. 2006 104: 234-239.

What is claimed is:

1. A compound 3-(1,1-dimethyl-allyl)-6-hydroxy-chromen-2-one, of general formula I. as given below 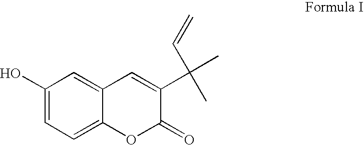
and its pharmaceutically acceptable salts thereof.

2. The compound according to claim 1, wherein the purity of the said compound by HPLC is more than 94%.

3. The compound according to claim 1, wherein the said compound is isolated from the plant Ruta graveolens.

4. The compound according to claim 1, wherein the said compound is useful as anti-inflammatory agent.

5. The compound according to claim 4, wherein the said compound causes in-vitro 25%, 40% and 70% nitric oxide (NO) inhibition at a concentration of 5, 10 and 20 μg/ml respectively.

6. The compound according to claim 4, wherein the said compound inhibits in-vivo inflammation (NO inhibition) in the range of 60% to 80%, at the effective dose of 40 mg/kg to 160 mg/kg body weight.

7. The compound according to claim 4, wherein the cytotoxicity of the said compound, is >85% at all the doses ranging from 20 to 100 μg/ml

8. The compound according to claim 4, wherein the said compound and its pharmaceutically acceptable salts are useful as a therapeutic agent.

9. The compound according to claim 4, wherein the said compound is useful in inhibiting inflammation.

10. The compound according to claim 4, wherein the said compound is useful in inhibiting inflammation by inhibition of nitric oxide (NO) production.

11. The compound according to claim 4, wherein the said compound is effective at very low concentration i.e., 5 μg/ml and less cytotoxic.

12. The compound according to claim 4, wherein the said compound and its pharmaceutically acceptable salts are useful for treatment of diseases selected from the group comprising of rheumatoid arthritis, systemic lupus erythrymatous and other inflammatory diseases in which nitric oxide plays an important role in pathogenesis or progression of the disease.

13. A pharmaceutical composition comprising compound 3-(1,1-dimethyl-allyl)-6-hydroxy-chromen-2-one, of general formula I, 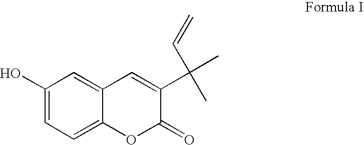
and its pharmaceutically acceptable salts thereof optionally along with a pharmaceutically acceptable carrier or an additive.

14. The pharmaceutical composition according to claim 13, wherein the said composition is in the form of powder, injectible fluid, syrup, capsule, or a tablet

15. A process for isolation of compound of claim 1, from the plant Ruta graveolens, wherein the said process comprising:
a. extracting the dried Ruta graveolens plant with an organic solvent at room temperature;
b. removing the solvent from the extract obtained from step a);
c. re-extracting the residue obtained from step (b) with solvents having comparable polarity to diethyl ether and removing the diethyl ether to obtain residue;
d. screening of the residue obtained from step (c) for inhibition of nitric oxide production;
e. purifying the active compounds from the residue obtained in step (d), using reverse phase-HPLC, and assaying all the peaks obtained for the inhibition of NO production;
f. characterizing and identifying the isolated purified compound obtained in step (e) using analytical techniques like ESI-MS/MS, MALDI-TOF, FT-IR and NMR.

16. The process for isolation of compound according to claim 15, wherein the solvent used for extraction is selected from the group consisting of alcoholic solvent preferably methanol, ketonic solvents preferably acetone and diethyl ether.
